(19) United States
(12) Patent Application Publication
(10) Pub. No.: US 2020/0261724 A1
(43) Pub. Date: Aug. 20, 2020

(54) COCHLEAR IMPLANT

(71) Applicant: TODOC CO., LTD., Seoul (KR)

(72) Inventors: Seung Ha Oh, Gangnam-gu (KR); Kyou-Sik Min, Suwon-si (KR); Doo Hee Kim, Seoul (KR)

(21) Appl. No.: 16/279,548

(22) Filed: Feb. 19, 2019

(51) Int. Cl. A61N 1/36; A61N 1/05; H04R 25/00

(52) U.S. Cl. CPC A61N 1/36038; A61N 1/0541; H04R 2225/021; H04R 2225/67; H04R 25/65

(57) ABSTRACT

A cochlear implant. An audio processor converts sound into an electrical signal. An outer coil unit transmits the electrical signal into the head of a user. An inner coil unit transmits and receives the electrical signal transmitted by the outer coil unit. A main body is connected to the inner coil unit to convert the electrical signal into an electrical stimulation signal. An electrode unit is connected to the main body and inserted into a cochlea to stimulate auditory nerves of the cochlea using the electrical stimulation signal. The outer coil unit is configured to surround an ear. The outer coil has a modified structure by which wearing sensation and implantation convenience are improved.

### BACKGROUND OF THE INVENTION

### Field of the Invention

The present disclosure relates generally to a cochlear implant and, more particularly, to a cochlear implant that can be conveniently attached without a magnet.

### Description of the Related Art

The information disclosed in the Background of the Invention section is only for the enhancement of understanding of the background of the invention, and should not be taken as an acknowledgment or as any form of suggestion that this information forms a prior art that would already be known to those skilled in the art.

A cochlear implant is a device fabricated to electrically stimulate remaining auditory nerves of a deaf person having a disease in the cochlea so that the deaf person can hear sound. The cochlear implant provides hearing ability to persons having medium or high-degree sensorineural hearing loss in one or both ears.

A related-art implant package for a cochlear implant operates by receiving power and electrical sound signals from an outer coil disposed outside of the body of a person, and generates electrical stimulation to stimulate the cochlea. Since a magnet is used for mounting to a location adjacent to the implant package, distortion occurs in an inspection system, such as a magnetic resonance imaging (MRI) scanner, which is problematic.

In this regard, Korean Patent No. 10-1609173 discloses a glasses-shaped cochlear implant, and Korean Patent No. 10-0735078 discloses a cochlear implant in which coils are magnetically connected.

However, the solutions of the related art have problems in that the coils are coupled using a magnet or the cochlear implant has a large volume. In addition, these solutions fail to disclose a configuration of connecting the coils without a magnet.

### RELATED ART DOCUMENT

Patent Document 1: Korean Patent No. 10-1609173

Patent Document 2: Korean Patent No. 10-0735078

### SUMMARY OF THE INVENTION

Accordingly, the present invention has been made keeping in mind the above problems occurring in the related art, and the present invention proposes a cochlear implant provided with an outer coil having a modified structure by which wearing sensation and implantation convenience are improved.

The object of the present disclosure is not limited to the aforementioned description, and other objects not explicitly disclosed herein will be clearly understood by those skilled in the art to which the present disclosure pertains from the description provided hereinafter.

In order to achieve the above object, according to one aspect of the present invention, a cochlear implant may include: an audio processor converting sound into an electrical signal; an outer coil unit transmitting the electrical signal into the head of a user; an inner coil unit transmitting and receiving the electrical signal transmitted by the outer coil unit; a main body connected to the inner coil unit to convert the electrical signal into an electrical stimulation signal; and an electrode unit connected to the main body and inserted into a cochlea to stimulate auditory nerves of the cochlea using the electrical stimulation signal, wherein the outer coil unit is configured to surround an ear.

In addition, the outer coil unit may include a plurality of turns of coil disposed adjacent to the inner coil unit, in a location between the ear and the head, to surround the ear.

The outer coil unit may include: an outer coil closely fitted to or attached to the ear or skin while surrounding the ear, with central portion thereof defining a space allowing the outer coil to be worn around the ear; and a coil cover covering the outer coil.

The outer coil may include a structure wound in a spiral, loop, or helical shape, an inner diameter and a number of turns of the outer coil are determined depending on physical characteristics of the ear and surroundings of the ear of the user. The outer coil may surround the ear in a polygonal shape included of linear segments connected at predetermined angles.

The outer coil may expand in a direction facing away from an external auditory canal and is disposed adjacent to the inner coil unit.

In addition, the inner coil unit may be located within an inner diameter of the outer coil and may be implanted in the head to be adjacent to a location below the skin.

According an embodiment of the present disclosure as set forth above, in the cochlear implant, the outer coil can be firmly mounted to the body of a user without a magnet used when providing bonding force between the outer coil and the inner coil inside of the body. When an inspection is performed using an inspection system, such as a magnetic resonance imaging (MRI) scanner, distortion in an image of a portion in which the cochlear implant is implanted can be advantageously minimized.

In addition, in the cochlear implant, the outer coil can be reliably mounted to the body due to a structural modification of the outer coil. The coil inside of the body can be advantageously implanted in a variety of locations depending on physical conditions of the user.

Furthermore, in the cochlear implant, the shape of the outer coil may be modified depending on physical conditions of the user and may be bent to be closely fitted to the skin, thereby improving the wearing sensation of the user having the outer coil attached thereto for an extended time.

Since the effects of the present disclosure are exerted by contents described herein, regardless of perception of the inventors, the above-described effects are merely specific examples of the effects obtainable from the present disclosure and should not be understood as all effects that the inventors have perceived or are obtainable from the present disclosure.

In addition, the effects of the present disclosure should be determined additionally on the basis of the complete description of the specification. Although not explicitly described, any effects that may be recognized by those skilled in the art to which the present disclosure relates, on the basis of the description of the specification, shall be regarded as the effects described herein.

### BRIEF DESCRIPTION OF THE DRAWINGS

The above and other objects, features and advantages of the present invention will be more clearly understood from the following detailed description when taken in conjunction with the accompanying drawings, in which:

FIG. 1 is a perspective view illustrating a cochlear implant according to an embodiment of the present disclosure;

FIG. 2 is a schematic view illustrating the cochlear implant illustrated in FIG. 1, implanted in the body of a user;

FIG. 3 is a schematic view illustrating the cochlear implant illustrated in FIG. 2, implanted in the body of a user;

FIG. 4 illustrates an application in which the outer coil of the cochlear implant illustrated in FIG. 1 is worn around an ear;

FIG. 5 is a side view illustrating a cochlear implant according to another embodiment of the present disclosure; and

FIGS. 6 and 7 illustrate positions of the inner coils when the cochlear implants illustrated in FIGS. 1 and 5 are used.

### DETAILED DESCRIPTION OF THE INVENTION

Hereinafter, a cochlear implant according to exemplary embodiments will be described, in relation to configurations, operations, and effects thereof, with reference to the drawings. For reference, in the drawings, components are omitted or schematically illustrated for the sake of convenience and clarity, and the size of each component may not reflect the actual size. Throughout this document, the same reference numerals and symbols will be used to designate the same or like components. In individual drawings, reference numerals of the same components will be omitted.

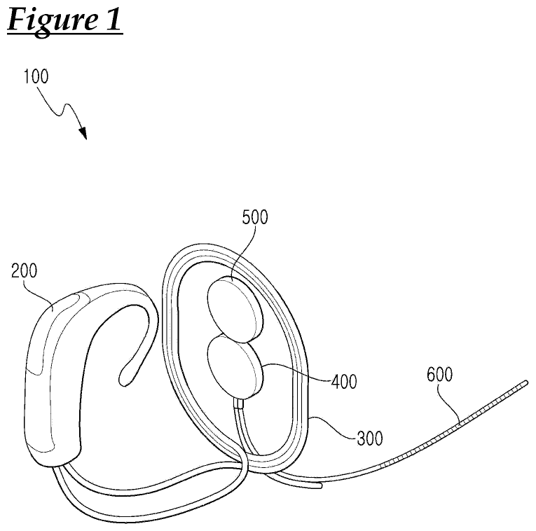

FIG. 1 is a perspective view illustrating a cochlear implant according to an embodiment of the present disclosure.

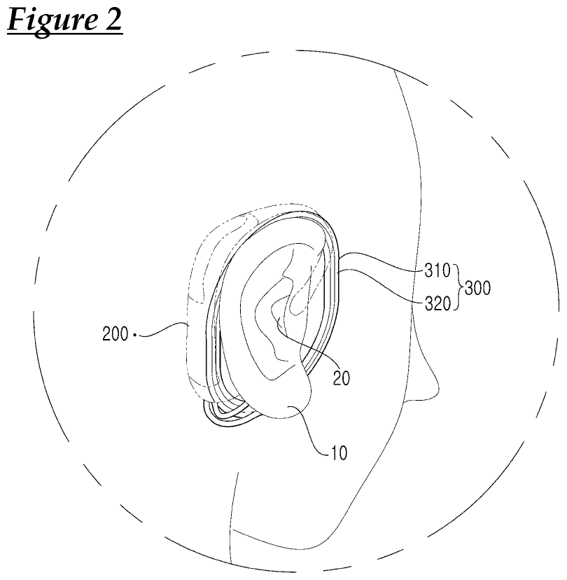

FIG. 2 is a schematic view illustrating the cochlear implant illustrated in FIG. 1, implanted in the body of a user.

As illustrated in FIGS. 1 and 2, a cochlear implant 100 includes an audio processor 200, an outer coil unit 300, an inner coil unit 400, a main body 500, and an electrode unit 600.

In the cochlear implant 100, the inner coil unit 400, the main body 500, and the electrode unit 600 are implanted in the body of a user, while the audio processor 200 and the outer coil unit 300 provide an extracorporeal device disposed outside of the body of the user. The outer coil unit 300 wirelessly transfers electrical sound signals to the inner coil unit 400, which are supposed to be transferred to auditory nerves of the cochlea.

However, since an outer coil unit of the related art disposed outside of the body uses a magnet to be attached to the body, in a location adjacent to the inner coil unit within the body, when an inspection is performed using an inspection system, such as a magnetic resonance imaging (MRI) scanner, a result of the inspection of a portion of the head in which the magnet is disposed is unclear, which is problematic.

In this regard, the cochlear implant 100 is fabricated such that the outer coil unit 300 surrounds an ear 10 or is closely fitted to or attached to the skin around the ear 10. Thus, the outer coil unit 300 can be advantageously fitted to the body of a user, in a location adjacent to the inner coil unit 400, without a risk of magnetic interference.

The audio processor 200 is configured to be fitted to a location adjacent to the ear 10, and serves to receive external sound using an internal microphone and convert the sound into electrical signals. A volume controller for adjusting the volume of the received sound may be provided on an outer portion of the audio processor 200.

The outer coil unit 300 is connected to the audio processor 200 to transmit the electrical signals, as well as power supplied by the audio processor 200, into the body of the user. The outer coil unit 300 may be configured to be worn on the ear 10, in a location between the ear 10 and the head, be closely fitted to the skin adjacent to the ear 10 by frictional force while surrounding the ear 10, or be attached to the skin adjacent to the ear 10 while surrounding the ear 10. In this manner, outer coil unit 300 may fitted to an outer portion of the body.

The outer coil unit 300 may include a spiral antenna coil, the entirety of which is covered with a flexible and pliable polymer material, or the surface of which is coated with a thin film, in order to prevent the coil made of a metal from contacting the skin. A surface of the outer coil unit 300, facing the skin, may have adhesion.

In addition, when the outer coil unit 300 is coupled to a portion of the audio processor 200 and the audio processor 200 is worn around and fitted to the ear 10, the outer coil unit 300 may be configured to be closely fitted to or attached to the skin around the ear 10.

The inner coil unit 400 is implanted in an inner portion of the head of the user. The inner coil unit 400 receives electrical signals or power, transmitted by the outer coil unit 300, or transfers the electrical signals or power to the main body 500 connected thereto.

The main body 500 operates using the power and converts the electrical signals into stimulation signals to stimulate the auditory nerves of the cochlea. The converted stimulation signals are output through the electrode unit 600 to stimulate the auditory nerves.

Specifically, the outer coil unit 300 includes an outer coil 310 and a coil cover 320.

The outer coil 310 may be configured in the shape of a cylinder or a plate to surround the ear 10. The outer coil 310 may form a circular surface to spirally surround the ear 10.

That is, the outer coil 310 may be in the shape of a ring or a washer. When the outer coil 310 is fabricated in the shape of a washer, the outer coil 310 may be closely fitted to or attached to the ear 10 or the skin 30, thereby being firmly mounted between the ear 10 and the skin 30.

The coil cover 320 is made of a flexible and elastic polymer material, with the surface thereof being soft. The coil cover 320 is configured to cover the outer coil 310, except for a central space portion, through which the ear 10 can pass. This can advantageously prevent the outer coil 310 made of a metal from contacting the body of the user and improve wearing sensation.

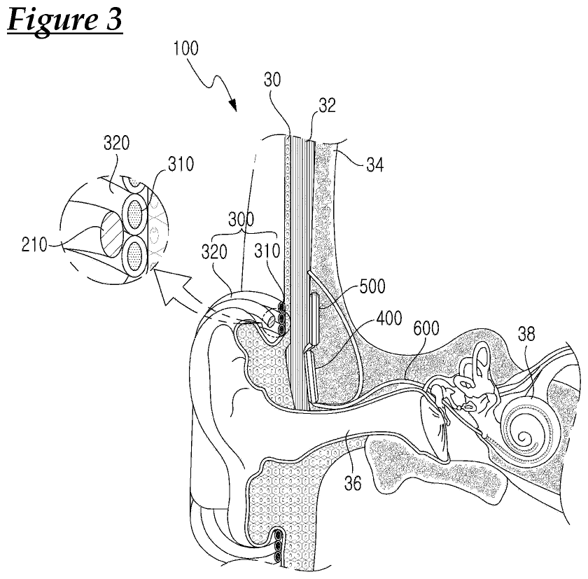

FIG. 3 is a schematic view illustrating the cochlear implant illustrated in FIG. 2, implanted in the body of a user.

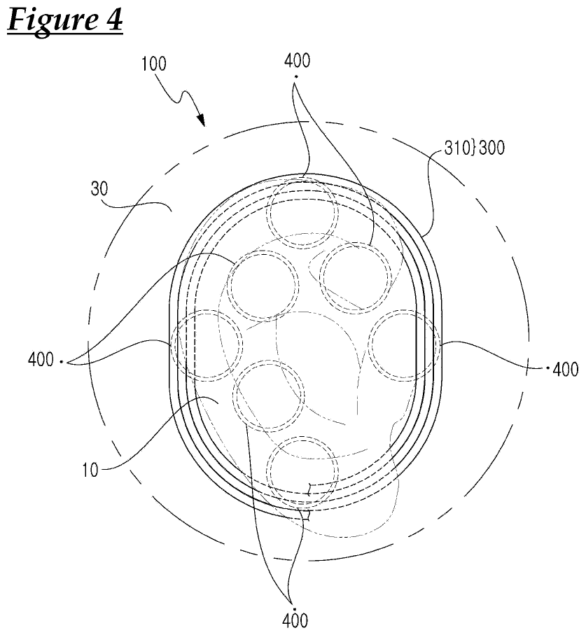

FIG. 4 illustrates an application in which the outer coil of the cochlear implant illustrated in FIG. 1 is worn around an ear.

As illustrated in FIGS. 3 and 4, the outer coil 310 have the shape of a spiral to surround the ear, and are arranged such that the surface of the outer coil 310 is closely fitted to the skin 30.

The coil cover 320 is configured to surround the outer coil 310, except for a central space surrounding the ear 10, and is disposed between the audio processor 200 and the skin 30 of the head in a space between the ear 10 and the skin 30.

An ear tip 210 provided on the top end of the audio processor 200 to surround a portion of the ear is closely fitted to the coil cover 320, in a location between turns of the outer coil 310 disposed within the coil cover 320. The ear tip 210 extends along a curved surface of the outer coil 310 to be closely fixed to or attached to a location between the ear 10 and the skin 30.

Next, the ear tip 210 is seated on and fixed to a space defined between the turns of the outer coil 310, and is closely fixed between the coil cover 320 and the ear 10. The outer coil unit 300 is disposed between the ear tip 210 and the skin 30 to advantageously improve fixing force and wearing sensation.

The coil cover 320 may be configured to coat the surface of the outer coil 310. In this case, the space between the turns of the outer coil 310 having a spiral shape surrounding the ear 10 may be adjusted as desired by the user.

In a position in which the ear tip 210 is tightly fitted to the skin 30 and the ear 10 through the space between the turns of the outer coil 310, the upper and lower portions of the ear tip 210 are fixed by the outer coil 310, so that the ear tip 210 can be worn on the body of the user when the space between the ear 10 and the skin 30 is narrow.

The inner coil unit 400 and the main body 500 are implanted inside of the skin 30 while being separated from or coupled to each other. The main body 500 is implanted in an incised portion of a bone tissue 34, in a location between a muscle 32 and the bone tissue 34.

In particular, the inner coil unit 400 is located within an inner diameter of the outer coil unit 300 to properly transmit electrical signals and power. The inner coil unit 400 may be implanted inside of the skin 30, in a location in which the outer coil unit 300 is closely fitted to the skin 30. The inner coil unit 400 may be implanted in a location inside of the skin 30 around the ear 10 and surrounded by the outer coil unit 300.

The inner coil unit 400 may be separated from the main body 500 and disposed between the skin 30 and the muscle 32. The inner coil unit 400 and the main body 500 are connected to each other via an electrically-conductive wire.

The electrode unit 600 extends through the bone tissue 34 to be inserted into the cochlea 38 of the internal ear, and a reference electrode connected to the main body 500 is grafted to a portion of the skin 30 or the muscle 32 adjacent to the main body 500.

Thus, sound received by the audio processor 200 is converted into electrical signals to be transmitted by the outer coil unit 300 to the main body 500. The electrical signals transmitted to the main body 500 are converted into electrical stimulation signals to stimulate the auditory nerves in the cochlea 38 through the electrode unit 600.

In addition, since neither the outer coil unit 300 nor the inner coil unit 400 uses a magnet, it is possible to minimize image portions corresponding to the outer coil unit 300 and the inner coil unit 400 being unclear when captured by a body inspection system, such as an MRI scanner, using magnetic fields.

In addition, since the ear tip 210 of the audio processor 200 worn on the ear is seated along the space defined between the outer coil 310, in a location between the outer coil 310 and the ear 10, there is an advantage in that the outer coil unit 300 can be reliably worn on the ear 10 without a magnet.

Referring to FIGS. 3 and 4, the inner coil unit 400 may be disposed inside of the skin 30, in any location within the inner diameter of the outer coil 310, and be implanted in a space between the skin 30 and the muscle 32 or a space defined between the muscle 32 and the bone tissue 34.

The inner coil unit 400 may be implanted to a variety of locations under the skin 30 around the ear 10, and thus it is possible to advantageously select a location in which the inner coil unit 400 can be reliably and conveniently implanted, depending on physical conditions of the user.

Specifically, the inner coil unit 400 may be implanted in a variety of locations inside of the skin 30 adjacent to the outer coil 310, along a direction in which the outer coil 310 extends, or may be implanted in a variety of locations inside of the skin 30 surrounding the canal of the ear 10 inside of a portion of the skin corresponding to the area of the outer coil 310.

That is, the inner coil unit 400 may be implanted such that the skin 30 is located between the outer coil 310 and the inner coil unit 400. Alternatively, the inner coil unit 400 may be implanted inside of the outer coil 310 such that the outer coil 310 and the inner coil unit 400 contact different portions of the skin 30, respectively.

In this case, the inner coil unit 400 may be disposed inside of the skin 30 so as not to move out of the outer coil 310. When it is necessary to dispose the inner coil unit 400 to be spaced apart from the ear 10 depending on physical conditions, the size of the outer coil 310 surrounding the ear 10 may be increased to increase locations in which the inner coil unit 400 can be implanted.

The inner diameter and the number of turns of the outer coil 310 surrounding the ear 10 may vary depending on physical conditions surrounding the ear 10. The inner diameter and the number of turns of the outer coil 310 may be changed such that the outer coil 310 and the inner coil unit 400 are located adjacent to each other, regardless of the location in which the inner coil unit 400 is implanted.

The outer coil 310 may be wound in the shape of a spiral, a loop, or a helix. Each the loop or helical outer coil 310 may allow the ear 10 to pass through the central space thereof and be worn on the ear 10, and the outer coil 310 may be bent toward the ear 10 or the skin 30, so that the outer coil 310 can be closely fitted to and reliably mounted to the ear 10 or the skin 30. The outer coil 310 may have an adhesive on the surface facing the skin 30 so as to be attached to the skin 30 using the adhesive.

In addition, the outer coil 310 may be implemented as one selected from among, but not limited to, a copper pipe, a copper wire, a copper strand, a pure platinum member, a platinum alloy member, and a gold member. It is possible to adjust the efficiency of transmission of electrical signals and power by changing the material of the outer coil 310 depending on the distance between the outer coil 310 and the inner coil unit 400.

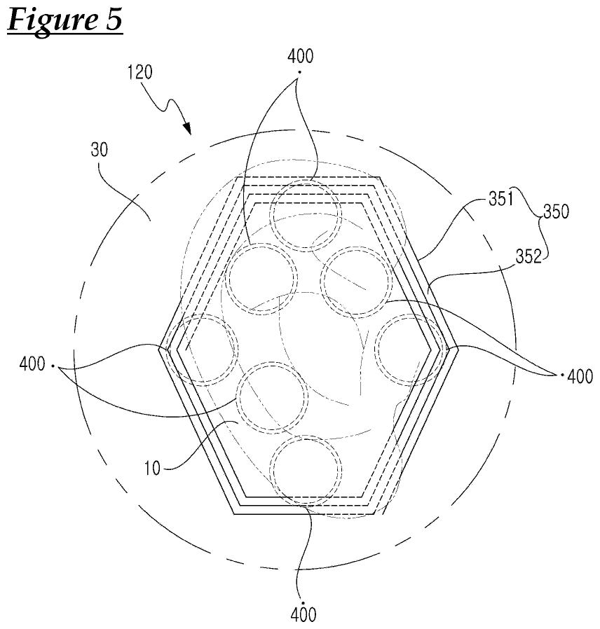

FIG. 5 is a side view illustrating a cochlear implant according to another embodiment of the present disclosure.

A cochlear implant 120 according to the present embodiment is substantially the same as the cochlear implant 100 illustrated in FIGS. 1 to 4, except for an outer coil unit 350 and an outer coil 351. Reference numerals and names will be used to designate the same components and repeated descriptions will be omitted.

As illustrated in FIG. 5, the cochlear implant 120 includes the outer coil unit 350.

The outer coil unit 350 is configured to surround an ear 10 of a user. The outer coil unit 350 has the shape of a polygon comprised of linear segments connected to each other at predetermined inclined angles. The lengths of the linear segments may be the same or be different from each other.

In addition, the outer coil unit 350 is configured to radially expand in a direction facing away from the outer auditory canal 36 of the ear 10, thereby making it easy to implant the inner coil unit 400.

The outer coil unit 350 includes a coil cover 352 in addition to the outer coil 351.

The outer coil 351 may have the shape of a polygon comprised of linear segments connected to each other at predetermined inclined angles, the linear segments having different lengths depending on physical conditions surrounding the ear 10 of the user. Alternatively, the outer coil 351 may have the shape of a polygon comprised of linear segments connected to each other at predetermined inclined angles, the linear segments having the same length.

A portion of the upper end of the outer coil 351 may be provided as a curved surface surrounding an upper portion of the ear 10 to increase the area of the outer coil 351 closely fitted to the upper portion of the ear 10 in a position in which the outer coil 351 is worn on the upper portion of the ear 10 and is closely fitted to the ear 10 or the skin 30 so as to be firmly mounted to the ear 10 or the skin 30.

In addition, the outer coil 351 may be attached to the skin 30 around the ear 10 via the adhesive on the surface thereof facing the skin 30.

The inner coil unit 400 may be implanted in a variety of locations inside of the skin 30 adjacent to the outer coil 351, along a direction in which the outer coil 351 extends, or may be implanted in a variety of locations inside of the skin 30 surrounding the canal of the ear 10 inside of a portion of the skin corresponding to the area of the outer coil 351.

That is, the inner coil unit 400 may be implanted such that the skin 30 is located between the outer coil 351 and the inner coil unit 400 or may be implanted in a location inside of the outer coil 351 such that the outer coil 351 and the inner coil unit 400 contact different portions of the skin 30.

In this case, the inner coil unit 400 may be disposed inside of the skin 30 so as not to move out of the outer coil 351. When it is necessary to dispose the inner coil unit 400 to be spaced apart from the skin 10 depending on physical conditions, the size of the outer coil 351 surrounding the ear 10 may be increased to increase locations in which the inner coil unit 400 can be implanted.

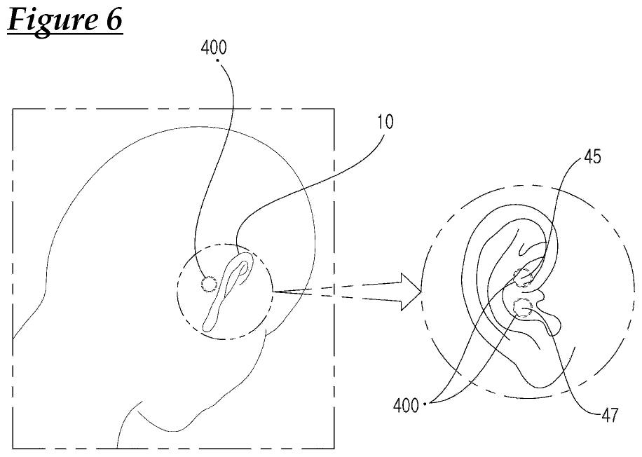

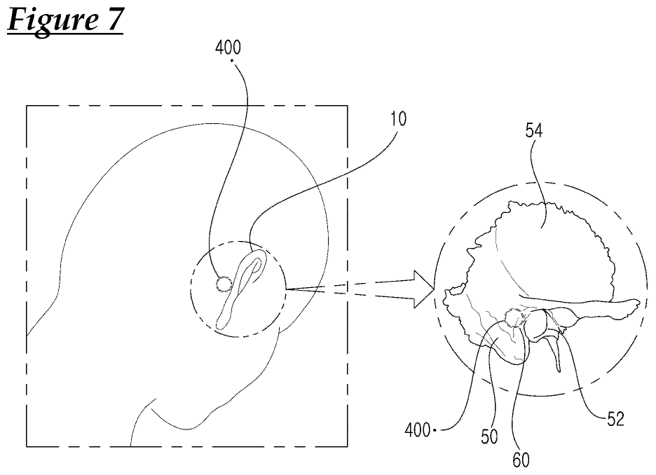

FIGS. 6 and 7 illustrate positions of the inner coils when the cochlear implants illustrated in FIGS. 1 and 5 are used.

As illustrated in FIG. 6, the inner coil unit 400 may be implanted under the skin at the rear of portions in which the cymba conchae 45 and the cavum conchae 47 of the ear 10 are located.

One of the outer coil units 300 and 350 is disposed to be closely fitted to the skin around the ear 10 while surrounding the ear 10, and the inner coil unit 400 is disposed inside of one of the outer coil units 300 and 350.

The inner coil unit 400 may be implanted in a removed portion of the cartridge of the ear 10, located under the skin at the rear of portions of the cymba conchae 45 or the cavum conchae 47, or may be disposed between the cartridge and the skin of the ear 10.

The cymba conchae 45 or the cavum conchae 47 are located on outer ends of the external auditory canal 36 illustrated in FIG. 3, and the inner coil unit 400 implanted under the skin at the rear of the cymba conchae 45 and the cavum conchae 47 is disposed to be inclined outward along the cartridge and the skin of the ear 10, which expand and extend from the cymba conchae 45 and the cavum conchae 47 toward the peripheral portions of the ear 10.

Thus, the inner coil unit 400 may be disposed in a variety of locations between the cartridge and the skin of the ear, inside of one of the outer coils 300 and 350. For the user having a physical structure in which a portion between the earflap and the cymba conchae 45 or the cavum conchae 47 is flat, the inner coil unit 400 may be disposed in a variety of locations between the earflap and the cymba conchae 45 or the cavum conchae 47.

As illustrated in FIG. 7, the inner coil unit 400 may be implanted in a location between the skin and a cartridge 60 below a temporal bone 54 between a mastoid 50 and a tympanic segment 52.

In this case, one of the outer coils 300 and 350 may be disposed to surround the ear 10 while being closely fitted to the skin outside of the mastoid 50 around the cartridge 60 below the temporal bone 54, in which the inner coil unit 400 is located, such that the inner coil unit 400 is located inside of one of the outer coils 300 and 350.

Specifically, a portion of one of the outer coils 300 and 350 is disposed to extend in the shape of a semicircle along the outer skin of the mastoid 50 and temporal bone 54, such that the inner coil unit 400 surrounds the cartridge 60 and the tympanic segment 52.

The inner coil unit 400 is fixed to the temporal bone 54, due to a portion of the cartridge 60 below the temporal bone 54 being removed, or is disposed inside of one of the outer coils 300 and 350, with the inner coil unit 400 and the cartridge 60 being attached to each other.

Although the foregoing exemplary embodiments of the present disclosure have been described with reference to the accompanying drawings, the embodiments described in the specification and the configurations illustrated in the drawings are merely best modes of the present disclosure, rather than representing all aspects of the scope of the present disclosure. It should be understood that a variety of equivalents and alternative embodiments could be made at a point in time at which the present application was filed. Accordingly, the foregoing embodiments shall be interpreted as being illustrative, while not being limitative, in all aspects. It should be understood that the scope of the present disclosure shall be defined by the appended Claims rather than by the foregoing embodiments, and that all of modifications and alterations derived from the definition of the Claims and their equivalents fall within the scope of the present disclosure.

It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

What is claimed is:

1. A cochlear implant comprising:
an audio processor converting sound into an electrical signal;
an outer coil unit transmitting the electrical signal into the head of a user;
an inner coil unit transmitting and receiving the electrical signal transmitted by the outer coil unit;
a main body connected to the inner coil unit to convert the electrical signal into an electrical stimulation signal; and
an electrode unit connected to the main body and inserted into a cochlea to stimulate auditory nerves of the cochlea using the electrical stimulation signal,
wherein the outer coil unit is configured to surround an ear.

2. The cochlear implant according to claim 1, wherein the outer coil unit comprises a plurality of turns of coil disposed adjacent to the inner coil unit, in a location between the ear and the head, to surround the ear.

3. The cochlear implant according to claim 2, wherein the outer coil unit comprises:
an outer coil closely fitted to or attached to the ear or skin while surrounding the ear, with central portion thereof defining a space allowing the outer coil to be worn around the ear; and
a coil cover covering the outer coil.

4. The cochlear implant according to claim 3, wherein the outer coil comprises a structure wound in a spiral, loop, or helical shape,
an inner diameter and a number of turns of the outer coil are determined depending on physical characteristics of the ear and surroundings of the ear of the user, and
the outer coil surrounds the ear in a polygonal shape comprised of linear segments connected at predetermined angles.

5. The cochlear implant according to claim 4, wherein the outer coil expands in a direction facing away from an external auditory canal and is disposed adjacent to the inner coil unit.

6. The cochlear implant according to claim 3, wherein the inner coil unit is located within an inner diameter of the outer coil and is implanted in the head to be adjacent to a location below the skin.
